(19) United States
(12) Patent Application Publication
(10) Pub. No.: US 2016/0051404 A1
(43) Pub. Date: Feb. 25, 2016

(54) Cold Compress for Therapeutic Cooling

(71) Applicants: Ramsey Joe CHOUCAIR, Dallas, TX (US); Scott Emil COLERIDGE, Belle Mead, NJ (US); Janet Lynn MARIANI, Dallas, TX (US); Edward KOFFEMAN, Midlothian, TX (US)

(72) Inventors: Ramsey Joe CHOUCAIR, Dallas, TX (US); Scott Emil COLERIDGE, Belle Mead, NJ (US); Janet Lynn MARIANI, Dallas, TX (US); Edward KOFFEMAN, Midlothian, TX (US)

(21) Appl. No.: 14/928,354

(22) Filed: Oct. 30, 2015

Related U.S. Application Data
(63) Continuation-in-part of application No. 13/252,529, filed on Oct. 4, 2011, now abandoned.
Application No. 13/252,529 is a continuation-in-part of application No. 11/539,020, filed on Oct. 5, 2006, now abandoned.

(51) Int. Cl. A61F 7/10; A61F 7/02

(52) U.S. Cl. CPC A61F 7/10; A61F 7/02; A61F 2007/108; A61F 2007/0219; A61F 2007/022; A61F 2007/0271; A61F 2007/0273

(57) ABSTRACT

A cold compress for therapeutic cooling that has a permanently sealed flexible elastic bag comprised of a water and water vapor impermeable film. The bag contains free-flowing spheres, each having a shell and containing a material having a freezing point appropriate to a cold compress temperature. The sealed bag is free of any fluid that causes bridging between the spheres to prevent free-flowing of the spheres. When cooled, the cold compress remains flexible and elastic; and the spheres remain free-flowing such that the cold compress can conform to a simply curved or compound curved surface of the human body to thereby present a heat transfer contact surface area. The absorbed body heat provides latent heat of liquefaction to frozen material inside the spheres such that a temperature of the cold compress is substantially maintained for a longer time.

### CROSS REFERENCE TO RELATED APPLICATIONS

This application is a continuation-in-part of co-pending U.S. patent application Ser. No. 13/252,529 filed on Oct. 4, 2011, which is in turn a continuation-in-part of U.S. Ser. No. 11/539,020 filed on Oct. 5, 2006.

### BACKGROUND OF THE INVENTION

1. Technical Field

The present invention relates generally to an external therapeutic cooling device that contains a cooling medium. More specifically, the invention includes a flexible bag filled with a plurality of free-flowing particulates such that the cooled bag and contents can be shaped to the human anatomy for use in therapeutic cooling of injured joints.

2. Description of Related Art

Cold compresses are commonly used to provide cooling therapy to patients preparing for or recovering from trauma such as surgery or injury. Such cooling can reduce swelling in bodily tissues.

Ways to cool bodily tissue are known in the art. One such example is an ice pack. Ice is well suited as a cooling medium due to its large latent heat of fusion. Because ice has a large latent heat of fusion, it can absorb a relatively large amount of heat before it begins to melt. This property has led to the wide use of ice as a cooling means, especially therapeutic cooling. Some ice packs are thick and inflexible plastic containers filled with water that becomes ice upon freezing. Such packs, resembling a closed book, are flat and rigid. Another type of ice pack comprises a flexible rubber package with a screw on lid into which ice chunks can be placed. These ice packs, however, have great drawbacks. One drawback is that the ice pack is heavy. Another more serious drawback is that the ice pack presents a limited cooling surface area. This is especially true if the ice pack is being applied to a patient's face. The flat and rigid ice pack is incapable of conforming to the contours of a patient's face. The flexible rubber ice pack can better conform to contours than the flat ice pack, but the potential surface area is limited to the size of the ice chunks. Even if the ice is broken into smaller pieces, the resulting surface area is insufficient because as the ice melts, the water drains toward the low part of the pack forming a pool. When water drains into such pools, the cooling surface area is greatly reduced, and as a result the cooling efficiency is also reduced. Thus, the ice pack is unsuitable for some therapeutic purposes.

Prior art attempts to obviate the problem of limited surface area have instituted packs that exhibit flexibility. Some packs consist of water and an antifreeze agent. The antifreeze agent prohibits the pack from freezing and thus makes the pack flexible. However, because the water is not frozen, one drawback is that the unfrozen pack fails to take advantage of the large heat of fusion of the ice. Consequently, although the unfrozen pack is flexible, it fails to offer the same cooling potential as an ice pack. Other packs consist of a plurality of chambers, each chamber being filled with water. When the pack is frozen, the pack is bent and the ice is broken to provide limited flexibility. Although such a chambered pack takes advantage of the properties of ice, it also lacks the ability to contour a patient's face or other areas as it provides only limited surface area. Other packs consist of a polymer and water mixture that turns into a gel. One disadvantage in these packs is the propensity of water molecules to clump together and freeze. These clumps can be broken, but flexibility and surface area is still lost.

Despite all the options provided by the prior art, many medical professionals still use a frozen bag of peas as a therapeutic cooling medium. Because each pea acts independent of the other peas, when a bag of peas is placed on a patient's face the free-flowing peas are able to rise and fall to match the contours of the patient's face and/or other anatomical structures. This ability to mimic the contours of the patient's face maximizes surface contact and as a result provides efficient and effective cooling. Despite its wide use, there are several disadvantages of using peas. One such disadvantage is that organic matter decomposes and emits an odor because of bacterial contamination. Such decomposition and bacterial contamination can result in additional perceptive problems from transmission of odor through the bag material or as a result of leaking as bags of peas often have leaking seals. Such leaking can also result in medical problems as any bacteria can potentially undesirably leak onto the skin.

Another disadvantage is that medical professionals often place a bag or rag over the bag of peas for sanitary purposes. This reduces both the heat transfer and the contouring ability of a bag of peas. Additionally, over time a bag of peas becomes unusable. This results from many iterations of freezing and thawing of the vegetable which causes the peas to lose the ability to retain water. When a pea has lost the ability to retain water, it loses its integrity and becomes mushy. As a result, the bag of peas is essentially an ice pack, which exhibits many of the disadvantages of the ice pack discussed above. The water, not being absorbed by the pea, can form bridges or clumps of ice. This undesirably reduces surface contact. Thus, a need exists for a therapeutic cooling medium that mimics the high surface area and effectiveness of a bag of peas while further reducing disadvantages of the prior art. Further, a need exists for a therapeutic cooling medium that permits the addition of a bacteriostatic or bactericidal agent that inhibits or destroys the growth of bacteria.

### SUMMARY

An exemplary embodiment of a cold compress used for therapeutic cooling comprises a permanently sealed flexible elastic bag comprised of a water and water vapor impermeable film. The bag has an interior volume containing a plurality of free-flowing spheres, where each of the plurality of free-flowing spheres has a surrounding outer layer and contains a material. The material has a freezing point of about 0° C., and the material absorbs latent heat to convert from a frozen state to a liquid state. The interior volume of the sealed bag is free of any fluid that freezes at about 0° C. to about −25° C. Thus avoiding potential bridging between the spheres that hinders the free-flowing of the spheres. When cooled to a safe cold temperature for use as a cold compress, such as about 0° C., the cold sealed elastic bag remains flexible and elastic, and the plurality of spheres remain free-flowing such that the cooled sealed bag can be manually pressed to a simply curved surface or a compound curved surface of the human body to conform to the shape of the simply curved or compound curve. As a result, the bag has a heat transfer contact surface area with the simply curved or compound curved surface of the body to absorb heat through the heat transfer contact surface from the simply curved or compound curved surface of the body. The absorbed heat provides latent heat to frozen material inside the plurality of spheres. Accordingly, until phase change from frozen solid to liquid is complete, temperature of the cold compress does not change (increase) significantly, but remains substantially constant. Thus, the temperature of the cold sealed flexible elastic bag is substantially maintained until a substantial proportion of the frozen material has liquefied.

Optionally, the bag may be configured with an integral or separate strap for ease of attachment to or around a body part of the human anatomy in need of treatment.

Optionally, the material inside each of the plurality of spheres may be selected from those materials that melt (undergo a phase change) at a temperature that is not harmful to human tissue when the cold compress is used. Thus, a non-limiting list of such materials includes water (ice melts at 0° C.), certain waxes, and a variety of other compositions that either occur naturally, or can be formulated based on physical properties. For reasons of costs, non-toxicity, and safety, water is preferred, but the invention is not limited to water as the sphere-filler material. When water or another material that expands upon phase change is used, the surrounding outer layer or “shell” of each of the plurality of spheres must be sufficiently elastic or strong to not rupture upon expansion.

Optionally, the material in the spheres may include a bactericide.

Optionally, the spheres may be in the size range from about 3 to about 10 mm. Mixed sizes may be used in any cold compress embodiment.

In another exemplary embodiment, the permanently sealed flexible elastic bag contains a substance that is liquid, non-reactive with the plurality of spheres, non-reactive with the flexible elastic sealed bag, and is not a solid in the range from about 0° C. to about −25° C. Thus in normal use, when cooled in a domestic freezer or hospital freezer for use as a cold compress, the substance remains liquid, albeit that viscosity and other physical properties may change, and does not cause bridging between the plurality of spheres, or only cause very minimal bridging so that the capability of the cold compress to conform to complex human body surfaces is not impaired. Thus, for a cold compress, the substance should have a lower freezing point than the freezing point of the material inside the spheres. Optionally, the substance inside the permanently sealed elastic flexible bag may be a gel, or a saline solution, or a like natural or synthetic composition having these physical characteristics. A saline solution is preferred for low cost and lack of toxicity, but other compositions may be preferred for better performance.

Optionally, the spheres may be coated to provide a “slick” non-stick surface, or the outer layer (“shell”) of each sphere can be made of a non-stick material, such as the polytetrafluoroethylene type polymers, commonly referred to as PTFE, and known by the trademark TEFLON (a trademark of DuPont de Nemours), and the like.

Optionally, the sealed flexible elastic bag may be compartmentalized by internal dividers into multiple compartments, each containing a plurality of spheres. This may facilitate in the effective wrapping or placing the bag on a simply or compound curved body part, in some circumstances. Each of the dividers may be perforated to allow fluid communication between the compartments to facilitate effective wrapping or placing the bag on a simply or compound curved body part. Alternatively, the dividers may be impermeable permitting no inter-compartment fluid flow. Optionally, at least some of the plurality of spheres are tethered to an inner surface of the compartments of the permanently sealed flexible elastic bag. Optionally, weighting elements are contained in the compartments of the bag for “weighted feel” and for other purposes. Optionally, the plurality of spheres are distributed in a pattern by fixed attachment to a flexible structure that is sized and configured to fit inside the compartments of the flexible elastic bag. Optionally, the flexible structure is “net-like” with a predetermined pattern of attachment points for each sphere or weighting element.

Another exemplary embodiment provides a cold compress used for therapeutic cooling comprising a permanently sealed flexible elastic bag comprised of a water and water vapor impermeable film. The interior volume of the sealed flexible elastic bag contains a structure comprised of a flexible plate having arrayed therein a plurality of geometric shaped cavities. Each of the cavities has a volumetric capacity that contains sealed therein a material having a freezing point. Thus, when the cold compress is in use, the material absorbs latent heat to convert from a frozen state to a liquid state. Further, when the cold compress is in use, the cold sealed flexible elastic bag remains flexible and elastic, and the plate remains flexible so that the cold compress conforms to a simply curved or compound curved surface of the human body and thereby presents a heat transfer contact surface area with the simply curved or compound curved surface of the body to absorb heat through the heat transfer contact surface area from the simply curved or compound curved surface of the body.

Optionally, at least some of the cavities is filled with a weighting element, such as metallic beads or the like. Optionally, the bag is divided internally into multiple compartments by dividers, and at least some of the compartments contains the structure comprised of a flexible plate having arrayed therein a plurality of geometric shapes.

In an exemplary embodiment there is provided a reusable cold compress, as a cooling medium by providing a hygienic disposable sheath sized for the sealed flexible elastic cold compress bag to slide into.

The above as well as additional features and advantages of the present invention will become more apparent in the following detailed description.

### BRIEF DESCRIPTION OF THE DRAWINGS

The novel features and characteristic of embodiments of the inventions are set forth in the appended claims. However, objectives and advantages may be better appreciated by reference to the following detailed description of illustrative exemplary and non-limiting embodiments, when read in conjunction with the accompanying drawings, wherein:

FIG. 1 illustrates a top cut-away view of the sealed flexible bag in accordance with an exemplary embodiment.

FIG. 2 illustrates a cross-sectional side view of a sphere in accordance with an exemplary embodiment.

FIG. 3 illustrates a side view of the exemplary sealed flexible bag depicted in FIG. 1 contouring to a user's face, which exemplifies a compound curved surface.

FIG. 4 illustrates an exemplary sheath that can be used to hold the exemplary sealed flexible elastic bag in accordance with one embodiment of the present invention.

FIGS. 5 A and B illustrate top and end views of an embodiment of a cold compress having multiple compartments, each containing a plurality of particulates (shown as spheres) that form the cooling medium, with or without a fluid substance in the compartments.

FIG. 6 illustrates a portion of an exemplary embodiment of a cold compress interior showing exemplary particulates tethered to the interior surface of the compress bag to maintain a particulate distribution.

FIG. 7 illustrates a portion of another exemplary embodiment of a structure, in this case net-like, to which particulates are attached to maintain a particulate distribution.

FIG. 8 illustrates a portion of an alternative embodiment of an exemplary structure that includes a series of geometric cavities, exemplified by truncated pyramids, filled with a heat absorbing material, arrayed on a flexible plate that is inserted into the bag as a container to form the cold compress.

Like reference numerals represent equivalent parts throughout the several drawings:
100—Cold Compress
101—Bag seals
102—Bag
200—Sphere or other-shaped particulate (hollow or solid). (In this disclosure, the exemplary embodiment is often a sphere, but the invention is not limited to using spheres.)
202—Sphere outer diameter
204—Sphere inner diameter
206—Sphere outer layer
208—Heat transfer fluid
209—Void space
210—Wall thickness
300—Cold compress contouring to a patient's face
400—Sheath

### DETAILED DESCRIPTION OF THE INVENTION

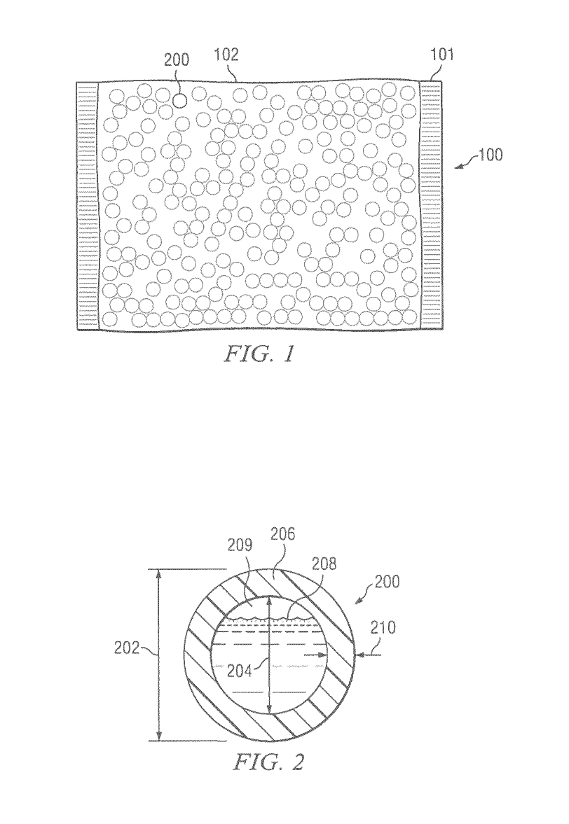

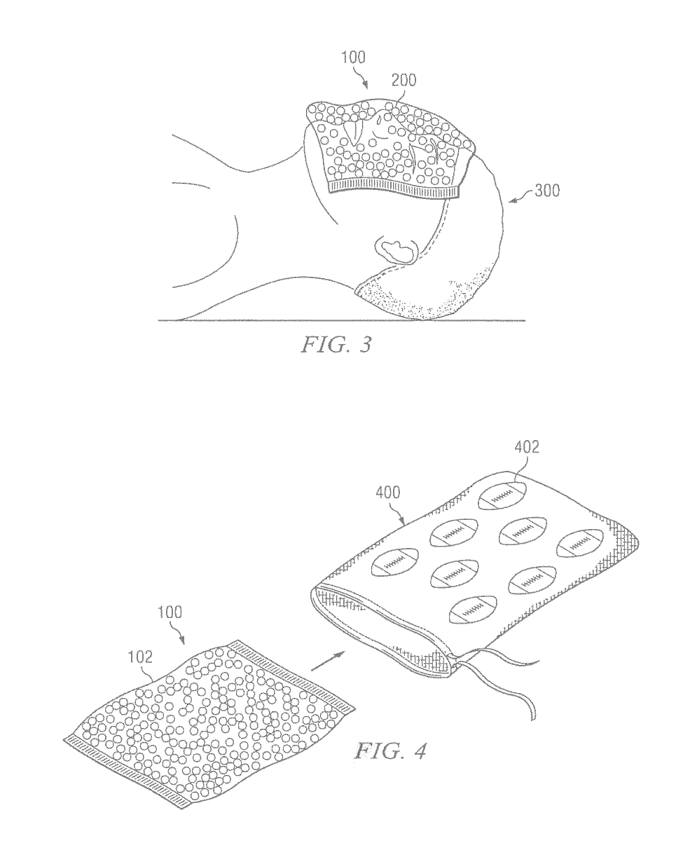

Referring now to the provided drawings, similar reference numerals represent the equivalent component throughout the several views of the drawings. FIG. 1 illustrates a top view of the sealed flexible bag in accordance with an exemplary embodiment of the present invention. FIG. 2 illustrates a cross-section of an exemplary embodiment of a sphere in accordance with the present invention. FIG. 3 illustrates a side view of an exemplary sealed flexible elastic bag cold compress, depicted in FIG. 1, contouring to a patient's face, which is a body part having compound curvature.

Referring to FIG. 1, the illustrated exemplary cold compress 100 comprises a plurality of free-flowing spheres 200 inside the sealed bag 102. As used herein, free-flowing is used to describe an independent object that is not connected or attached to another object and that is free to flow relative to its cohort (other spheres, in this example) and to respond independently to pressure and gravity. The cold compress 100 is depicted as having two transverse seals 101, which are permanent seals, such as can be produced by heat sealing, for example, or adhesives, or other known methods of sealing the material of the bag. Thus, the filled bag can be made by methods known in the art such as with vertical form fill and seal machines or other suitable method.

In an exemplary embodiment, the cold compress 100 may comprise two sheets of overlapping film that are sealed along each of four peripheral edges. The permanently sealed bag 102 useful in the present application is not limited by the number of seals, or by the method of manufacture. In an embodiment, the cold compress 100 can be transparent and seen to enclose the free flowing spheres 200.

In an exemplary embodiment, the cold compress 100 is configured and sized to wrap around a simple or compound curvature of a human body part and being held in place by attaching to itself, or another object, by VELCRO, mechanical clips, tape, or any other suitable means. In an exemplary embodiment, the cold compress 100 includes an affixing strap (not shown) that can be used to fasten the cold compress 100 to an anatomical structure. The affixing strap can wrap around a body member and affix to itself or to a portion of the cold compress 100. The size and shape of the cold compress 100 can be varied depending on the application. For example, if the cold compress 100 is to be applied to a patient's face, the bag 102 can have a slightly rectangular shape and be about the size of a sheet of paper (about 22 cm by 28 cm). Likewise, if an ankle is to be treated then the cold compress 100 could be more elongate for ease of wrapping around the entire ankle yet wide enough to cover only the ankle.

The bag 102 can comprise any flexible material that conducts heat and preferably has barrier properties as to water (liquid) and water vapor. Elasticity of the bag material is also desirable for ease of stretching around compound curvatures, and to allow the bag to return to its original “as manufactured” shape. A bag that has heat insulating properties (very low thermal conductivity) would decrease the effectiveness of the invention since body heat has to pass through the bag to the cooling medium inside the bag.

The bag 102 is preferably made of a material that has barrier properties to water and water vapor. Barrier properties are preferred because if moisture was allowed to enter the bag, the formation of ice bridges between particulates, such as spheres, within the bag causes hard clumps that can inhibit flexibility and contouring ability of the bag to conform to simple or compound anatomical curves. This decreases the contact surface area between the cold compress bag and the body part being cooled. Undesirable moisture in the bag can bond to or coat the spheres and, when frozen, form hardened inflexible frozen clumps of spheres. Consequently, in an exemplary embodiment, the bag 102 comprises water and water vapor properties sufficient to avoid clumping of the cooling medium, such as spheres, within the bag. As used herein, sufficient bag water barrier properties is defined as a bag having a water vapor transmission rate of less than about 20 g/mill/645.16 sq. cm (100 sq. in.)/day at 37.8° C. (100° F.) and 90% relative humidity with a preferable rate of less than about 2 g/mill/645.16 sq. cm (100 sq. in.)/day at 37.8° C. (100° F.) and 90% relative humidity.

The bag material must also be flexible at temperatures lower than the freezing temperature of water (0° C.) as well as temperatures greater than about 38° C. (body temperature). The bag should not become rigid and inflexible at lower temperatures at which the cold compress will be used as this will decrease the effectiveness of the cold compress. Likewise, the bag cannot melt or stretch inelastically at temperatures around body temperature (−38° C.). In one embodiment of the invention, the bag 102 is made from flexible films known in the art. The flexible film may be selected from polymers and polymer composites, such as but not limited to: high density polyethylene, medium density polyethylene, low-density polyethylene, polycarbonate, polypropylene, linear low density polyethylene, polyethylene terephthalate, polyvinyl chloride, polystyrene, polyurethane, polycarbonate or other suitable material, having the properties described herein.

In an exemplary embodiment, the bag 102 is made from other suitable materials or material composites, including, but not limited to, cloth-like materials such as micro-fibers, nylon, cotton, GORE-TEX, polyester blends, interwoven textiles and water-resistant paper such as waxy paper.

Referring to FIG. 2, the exemplary particulate is depicted as a sphere 200 with an outer diameter 202 and an inner diameter 204. In one embodiment, the outer diameter 202 (or sphere “size”) ranges from between about 2 mm to about 25 mm, more preferably between about 3 mm to about 10 mm, and with a most preferred diameter of about 8 mm. The inner diameter 204 is a function of the thickness 210 of the sphere outer layer 206. The inner diameter 204 is equal to the outer diameter 202 less twice the wall thickness 210 of the sphere 200. Any suitable wall thickness 210 can be used. All things being equal, a thinner wall thickness allows for an increased rate of heat transfer since the wall provides some barrier, however small, to heat transfer through it. Accordingly, wall thickness is a factor in designing for a desired rate of heat transfer. Different values for inner 204 and outer diameters 202 can also be selected depending on the desired size of the sphere 200. Generally, it might be expected that smaller spheres would yield greater surface area for heat transfer and more contourability of the cold compress but might incur higher manufacturing costs.

The particulate outer layer, such as the sphere outer layer 206 can be made of any material that has sufficient water and water vapor barrier and heat transfer properties. The sphere outer layer 206 should conduct heat, and should be water tight. The sphere outer layer 206 should act as a sufficient barrier to water vapor. As used herein, sufficient sphere barrier properties is defined as a sphere outer layer 206 having a water vapor transmission rate of less than about 20 g/mill/645.16 sq. cm (100 sq. in.)/day at 37.8° C. (100° F.) and 90% relative humidity with a preferable rate of less than about 2 g/mill/645.16 sq. cm (100 sq. in.)/day at 37.8° C. (100° F.) and 90% relative humidity. In an exemplary embodiment, the sphere outer layer 206 has a low coefficient of expansion. The reason for this is that if the outer layer 206 shrinks or expands with temperature at a much different rate than the fluid 208 it contains, then the sphere could crack or split. In alternative embodiments, the material of the sphere outer layer (“shell”) is elastic and expands and contracts as its contents freeze or liquefy.

In another exemplary embodiment, the sphere outer layer 206 comprises a non-stick material. As used herein, a non-stick material is one that does not bond or stick to itself so that the spheres remain free-flowing. If the sphere outer layer 206 was made of such a material (e.g., a gel) then the spheres would tend to cluster, reducing surface area and heat transfer. In one embodiment of the current invention, the outer sphere layer 206 is selected from one or more polymer or composites selected from, low, medium, or high density polyethylene, polypropylene, linear low density polyethylene, polyethylene terephthalate, polyvinyl chloride, polystyrene, polyurethane, or other suitable material. Other suitable non-stick, water tight materials can include, but are not limited to, metals, or metal composites, such as aluminum or steel, or a silica-based material such as fiberglass. In another embodiment the outer sphere layer 206 comprises glass, titanium, or brass.

Alternatively, in one embodiment, the outer sphere layer 206 is coated with a non-stick material such as a fluoropolymer such as polytetrafluoroethylene (PTFE) or a silicone-based coating comprising silicone resins, elastomers, oils or silicone glazes to help ensure the spheres are free-flowing.

In one embodiment, the sphere 200 is partially filled with a fluid 208 that is liquid at ambient conditions. Because of convenience and the high latent heat of fusion of H₂O, in one embodiment of the invention the fluid 208 comprises water. The water can be then be frozen into ice. Using ice permits the user of the instant invention to absorb a large amount of heat at substantially constant cold compress temperature before the ice begins to melt to water, and the cold compress temperature begins to increase gradually. Additionally, because of the availability of freezers to users of the cold compress, the temperature required to make ice, 0° C., is very achievable and convenient.

The conduction of heat is expressed by the mathematical formula below:

Q=cmΔT

Where Q=Heat Conducted or Heat Transferred
c=specific heat of the material conducting the heat
m=mass of the substance conducting the heat
ΔT=the temperature difference between the two mediums where heat transfer is taking place.

A material's heat capacity, denoted as “c” in the equation above, is quantified by the amount of heat energy required to raise the temperature of the material by a certain amount, such as 1° C.

In an exemplary embodiment, a fluid having a relatively high heat capacity is used. Thus, the fluid does not freeze at the temperature of use of the cold compress, but remains a fluid inside the spheres or other shaped particulates, and absorbs heat without phase change. The fluid 208 should have a high specific heat, greater than about 1 J/gram/Kelvin at 25° C. (constant pressure), and preferably greater than about 4 J/gram/Kelvin at 25° C. (constant pressure) in the liquid phase. Water in the liquid form exhibits a higher specific heat or heat capacity than water in the solid form (ice). For example, the heat capacity of water is 4.187 kJ/kg K and the heat capacity of ice is 2.108 kJ/kg K. Accordingly, faster heat transfer may be possible if liquid water is utilized.

One advantage of embodiments of the present invention is that, because the fluid 208 is placed into water-tight spheres, the fluid 208 can comprise coolants such as gels that would otherwise be undesirable because of the tendency of such gels to stick to one another. Thus, a fluid can be selected to maximize the amount of cooling time provided by the cold compress 100 of the present invention.

The driving force, denoted as “ΔT” in the equation above, is the temperature difference between the temperature of a patient's body part and the temperature of the bag material in communication with the body part. The driving force can be increased by supplying a colder medium in communication with the body part. One simple way to accomplish this is to add salt or an antifreeze solution to the water solution, thus lowering the freezing point. Depending on the concentration of the salt-water mixture, temperatures as low as −21° C. (for NaCl) can be reached without the mixture freezing. Further, because the spheres are water tight, and are further in a sealed flexible bag, any fluid used is less likely to leak and come into contact with a user than many prior art embodiments. Consequently, heat transfer fluids that may otherwise not be advisable for use can be used in accordance with various embodiments of the present invention.

As further clarification, one objective of one embodiment of the present invention is to provide a cold compress that can transfer at least as much heat (Q) from an anatomical member as a similar sized bag of frozen peas. As shown by the above formula, this can be achieved by using heat transfer fluids with high “c” values and/or by increasing the driving force (ΔT). Consequently, in one embodiment, the driving force is maximized by using a heat transfer fluid that does not freeze in a standard freezer, e.g., at temperatures lower than −22° C. Thus, in one embodiment, the heat transfer fluid comprises an antifreeze solution. This can permit a larger driving force to be utilized.

Alternatively, in an exemplary embodiment, it may be desirable to take advantage of the latent heat of fusion provided by a frozen fluid and a larger driving force than would be provided by water alone. Thus, in an exemplary embodiment, the heat transfer fluid comprises a chemical mixture to decrease the freezing point of the heat transfer fluid, but that permits the heat transfer fluid to freeze in a standard freezer, e.g., at temperatures higher than −22° C. The chemical mixture can be a salt (MgCl, NaCl, etc) added to water or any other suitable mixture that results in a freezing point of higher than −22° C.

In an exemplary embodiment, the volume of fluid 208 to be filled in each sphere 200 may depend on the expansion coefficient of the fluid 208 and the outer sphere layer material 206. Determination of the amount of fluid 208 to fill a given sphere size is within the knowledge of one skilled in the art. In this embodiment the sphere 200 is only partially filled with liquid 208 at room temperature. When partially filled, the sphere 200 has sufficient void space 209 within it to allow for liquid expansion at reduced temperatures. This will prevent the sphere from cracking or splitting due to the expansion pressure of the fluid 208. For example, it is known that water expands in volume by about 10% when frozen. As a result, in this embodiment the sphere 200 has a void space that occupies about 10% or more of the volume of the water. Depending on the expansion coefficient of the fluid 208 and the material used in manufacturing the sphere outer layer 206 the void space 209 can occupy up to about 50% of the total sphere volume, with a preferred void space volume of less than about 25% and most preferably between about 5% and about 15% of the total sphere volume as calculated by the sphere inner diameter 204. This allows the water to expand when frozen, and not split or crack the outer sphere layer 206. In another exemplary embodiment, the sphere 200 is completely filled with fluid. In such an embodiment, the sphere outer layer 206 is made of a flexible material that can expand (i.e. it is elastic) with the fluid 208. This flexible elastic material allows the fluid to expand without causing the sphere to crack or split. In one embodiment, the sphere thickness 210 is adjusted according to the level of fluid placed into the sphere 200.

In an exemplary embodiment, the spheres 200 may be manufactured on a machine called a “Blow-fill-seal Machine.” Such machines are known in the art and commonly used for the aseptic packaging of pharmaceuticals. The spheres can be made as follows: First, the outer sphere layer 206 is formed by extruding the material around a mold and blowing air into the mold to form the bulk of the sphere 200. Next, a measured dose of fluid 208 is injected into the partial sphere 200. Finally, the sphere 200 is capped or sealed on top.

Other suitable machines or methods can be used to manufacture the spheres 200. For example, U.S. Pat. Nos. 5,254,379 and 6,532,947 disclose methods of filling a substantially spherical object with a fluid. While these patents are directed towards paintballs, those skilled in the art will understand that such methods can be adapted to make the spheres of the present invention. Other suitable machines or methods such as vacuum form molding can also be used.

While one embodiment describing spheres 200 filled with a fluid has been disclosed, in another embodiment a solid object can be utilized. For example, the sphere 200 can comprise glass beads. These solid beads operate as previously described. For example, these solid beads are frozen or cooled to lower temperatures. When applied, heat is transferred from the object to be cooled to the solid beads. Other beads such as titanium beads, brass beads, copper beads, and other such beads which offer desirable heat transfer properties can also be suitably used. These include, but are not limited to, other metals and ceramics which have a high specific heat such as aluminum.

Referring to FIG. 3, the cold compress or therapeutic bag 100 is shown contouring a patient's face. Given that the spheres are small in size, the surface area and therefore surface contact between the cold compress 100 and the patient's face is maximized. Additionally, because sphere outer layer 206 is made from a non-stick material, clumps and clusters are avoided. This allows individual, free-flowing spheres to rise and fall with the contours of, for example, a human face. Thus, in operation, the instant invention mimics the successful utilization of a bag of peas. However, the current invention, unlike a bag of peas, can be reused many times without decreased efficiency by simply re-freezing the bag. Further, undesirable odors are avoided that can occur from the chemical breakdown of peas. Additionally, in one embodiment, the instant invention has a strap or other applicable device that secures the bag 102 in place. This eliminates the need for the patient to hold the bag in place.

In an exemplary embodiment, to minimize heat absorption from the environment, and to promote heat absorption from the body part to which it is applied, the cold compress is provided with a heat insulating side facing the environment, and a heat conduction side interfacing with the body part. Thus, the cold compress would be useful (i.e. “cold”) for a longer time before it needs to be cooled again in a freezer. Heat insulation can be achieved by selection of bag sidewall material and thickness, and by adding a highly reflective coating or film of a metal, such as aluminum. Such a coating or film will also readily indicate to the user which side should face the surrounding environment in use, and which should be placed on the body part.

In an exemplary embodiment, the cold compress 100 is placed into a sheath that is sized such that the cold compress 100 can be placed inside the sheath. FIG. 4 illustrates an exemplary sheath 400 that can be used to hold the flexible bag in accordance with one embodiment of the present invention. The exemplary sheath 400 can be a configured in any suitable manner and its configuration can emulate a pillowcase, a sock, a sock-like sheath having a drawstring, etc. The sheath 400 can be of any suitable material, and is not limited to those disclosed above as suitable for the bag 102. In an exemplary embodiment, the sheath comprises a decorative design 402. For example, the decorative design 402 can include a picture of a football and may be popular for application of the cold compress 100 to sports injuries (e.g., an ankle injury).

Use of the sheath facilitates reusability of the cold compress by the user and/or health care providers. For example, in one embodiment, before the cold compress 100 of the present invention is used on a patient, a sheath 400 is applied to the outer portion of the bag 102. When the cold compress 100 is subsequently removed, the sheath can be removed and discarded and the cold compress 100 can be optionally washed and/or disinfected and placed into a freezer for re-use. The sheath can also be made from many of the same materials disclosed above to make the bag 102.

In one embodiment, the sheath comprises a device for attaching the bag 102 to an anatomical structure. The same devices discussed above, such as VELCRO, mechanical clips, tape, or any other suitable device so long as the cold compress 100 helps to fasten the bag 102 to an anatomical structure.

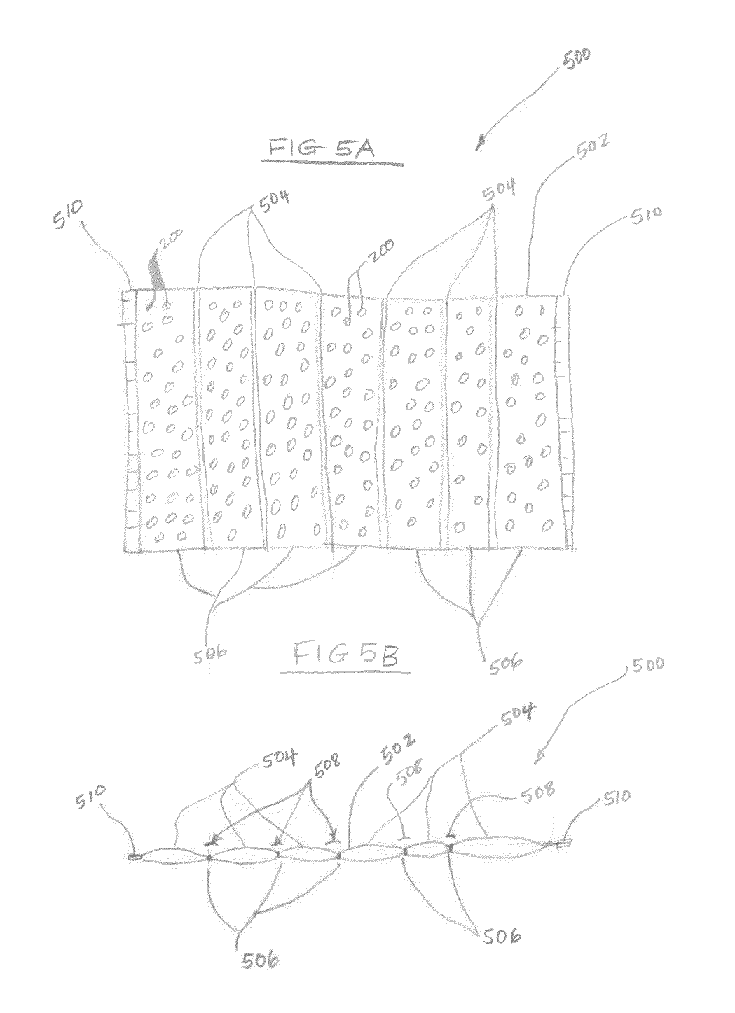

In an exemplary embodiment illustrated in FIGS. 5A and 5B, the cold compress 500 includes a bag 502 that is separated internally by dividers 504 into a series of compartments 506, and sealed permanently at each end with end seals 510. Each compartment 506 is at least partially filled with a particulate 200, such as a sphere, that contains the cooling medium, for example, but not limited to frozen water. Note that the “particulates” can encompass any convenient shape and is not limited to spherical shapes, although these are convenient, especially if these are formed with an outer shell, and are filled with a heat transfer medium for absorbing heat from a human body part. The compartments may vary in size, depending upon the contoured human body part it is intended to conform to as a cold compress. Thus, for wrapping around a knee, some central areas that rest on the knee may have larger compartments, and those areas that wrap around the sides to the back of the knee may have smaller (narrower in the illustrated embodiment) compartments for ease of contouring. Further, the compartments may extend longitudinally or transversely, depending upon the intended use of the cold compress. As illustrated in FIG. 5B, the exemplary illustrated cold compress bag 502 may have flexibility around the regions 508 where there are dividers 504. This added flexibility facilitates wrapping around a curved body part, whether of simple or compound curvature. In addition, the space around the spheres in the compartments may be at least partially filled with a fluid, and the compartments may not each be filled with an equal amount of fluid or spheres. Thus, by selecting compartment dimension, number of compartments, divider configuration (for added flexibility), number of particulates (illustrated as spheres) per compartment and size of particulates, and the amount of fluid substance, if any in each compartment along with the spheres, the configurability of the cold compress to more closely conform to both simple and compound body parts may be facilitated. In a further exemplary embodiment, the dividers may be porous and permit fluid communication with some or all adjacent compartments to further facilitate configurability of the cold compress to both simple and compound body parts. Thus, the dividers may be of textile fabric or porous polymeric material, that will readily flex and that is durable under conditions of use.

An exemplary embodiment of a cold compress used for therapeutic cooling comprises a permanently sealed flexible elastic bag comprised of a water and water vapor impermeable film. The bag has an interior volume containing a plurality of free-flowing spheres, where each of the plurality of free-flowing spheres has a surrounding outer layer and contains a material. The material has a freezing point of about 0° C., and the material absorbs latent heat to convert from a frozen state to a liquid state. The interior volume of the sealed bag is free of any fluid that freezes at about 0° C. to about −25° C. Thus avoiding potential bridging between the spheres that hinders the free-flowing of the spheres. When cooled to a safe cold temperature for use as a cold compress, such as about 0° C., the cold sealed elastic bag remains flexible and elastic, and the plurality of spheres remain free-flowing such that the cooled sealed bag can be manually pressed to a simply curved surface or a compound curved surface of the human body to conform to the shape of the simply curved or compound curve. As a result, the bag has a heat transfer contact surface area with the simply curved or compound curved surface of the body to absorb heat through the heat transfer contact surface from the simply curved or compound curved surface of the body. The absorbed heat provides latent heat to frozen material inside the plurality of spheres. Accordingly, until phase change from frozen solid to liquid is complete, temperature of the cold compress does not change (increase) significantly, but remains substantially constant. Thus, the temperature of the cold sealed flexible elastic bag is substantially maintained until a substantial proportion of the frozen material has liquefied.

Optionally, the bag may be configured with an integral or separate strap for ease of attachment to or around a body part of the human anatomy in need of treatment.

Optionally, the material inside each of the plurality of spheres may be selected from those materials that melt (undergo a phase change) at a temperature that is not harmful to human tissue when the cold compress is used. (Applying a very cold compress for a long time may cause “cold burn” and tissue damage. In general, the colder the compress the shorter the time for cold burn. Accordingly, a cold compress should have a temperature tolerable for human tissue when applied for the time periods specified). Thus, a non-limiting list of such materials includes water (ice melts at 0° C.), certain waxes, and a variety of other compositions that either occur naturally, or can be formulated based on physical properties. For reasons of costs, non-toxicity, and safety, water is preferred, but the invention is not limited to water as the sphere-filler material. When water or another material that expands upon phase change is used, the surrounding outer layer or “shell” of each of the plurality of spheres must be sufficiently elastic to not rupture upon expansion.

Optionally, the material in the spheres may include a bactericide.

Optionally, the spheres may be in the size range from about 3 to about 25 mm. Mixed sizes may be used in any cold compress embodiment. In addition, to add a “dead weight feel” to the cold compress, weighting elements in any of a variety of shapes, but most commonly spheres, that may be solid metallic, may be added to the bag contents to increase mass and develop that feel. The added mass may also facilitate conformance of the compress to body curvatures.

In another exemplary embodiment, the permanently sealed flexible elastic bag contains a substance that is liquid, non-reactive with the plurality of spheres, non-reactive with the flexible elastic sealed bag, and is not a solid in the range from about 0° C. to about −25° C. Thus in normal use, when cooled in a domestic freezer or hospital freezer for use as a cold compress, the substance remains liquid, albeit that viscosity and other physical properties may change, and does not cause bridging between the plurality of spheres, or only cause very minimal bridging so that the capability of the cold compress to conform to complex human body surfaces is not impaired. Thus, for a cold compress, the substance should have a lower freezing point than the freezing point of the material inside the spheres. Optionally, the substance inside the permanently sealed elastic flexible bag may be a gel, or a saline solution, or a like natural or synthetic composition having these physical characteristics. A saline solution is preferred for low cost and lack of toxicity, but other compositions may be preferred for better performance. The substance may serve one or all of several functions, such as transferring heat absorbed through the bag film to the spheres, permit more even cooling of the skin, and adding mass to the cold compress to aid in conformance to curved body parts.

Optionally, the spheres may be coated to provide a “slick” non-stick surface, or the outer layer (“shell”) of each sphere can be made of a non-stick material, such as the polytetrafluoroethylene type polymers, commonly referred to as PTFE, and known by the trademark TEFLON (a trademark of DuPont de Nemours), and the like.

Optionally, the sealed flexible elastic bag may be compartmentalized by internal dividers into multiple compartments, each containing a plurality of spheres. This may facilitate in the effective wrapping or placing the bag on a simply or compound curved body part, in some circumstances. Each of the dividers may be perforated to allow fluid communication between the compartments to facilitate effective wrapping or placing the bag on a simply or compound curved body part. Alternatively, the dividers may be impermeable permitting no inter-compartment fluid flow.

An issue that may arise in cold compresses whether the bag is fluid filled or not, is the tendency of spheres or other particulates to migrate under gravity (or other forces such as buoyancy) to cluster in parts of the cold compress thereby impairing heat absorption performance.

In order to address the migration issue, FIG. 6 illustrates a section of an exemplary embodiment of a cold compress 600 interior showing exemplary particulates 200 tethered by “strings” 205 to the interior surface of the compress bag to maintain the particulate distribution within the cold compress. The strings 205 may be formed along with the spheres 200, and might then be of the same material as the sphere shells. The strings are then adhered or sealed to the interior surface of the bag in a predetermined pattern. Alternatively, the strings 205 may be formed along with the bag interior surface, and might then be of the same material as the bag itself. In this case, the spheres or other-shaped particulates 200 are then adhered or sealed to the strings. The particulates 200 may be filled with a heat absorbing fluid or solid. The particulates 200 may be solid, such as metallic to provide weighting. The particulates 200 may be a mixture of these, and of mixed sizes, to provide a predetermined weighting and heat absorption capacity. This exemplary embodiment may also be used in conjunction with the multiple compartment exemplary embodiments described herein.

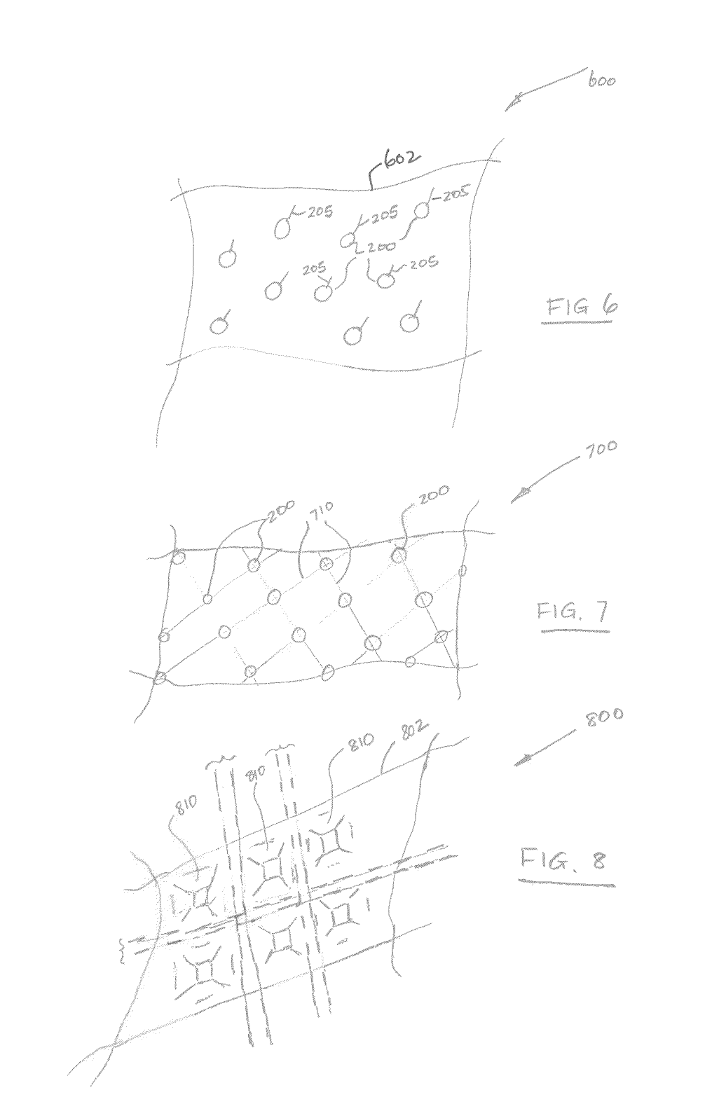

As an alternative, FIG. 7 illustrates another exemplary embodiment of a structure 700, in this case net-like, having crisscrossing filaments 710 to which particulates 200 are attached at predetermined intervals to maintain a particulate distribution. The net-like structure is flexible, and is placed inside the cold compress bag (see FIG. 1, bag 102) where it is affixed in place. The bag interior may be otherwise at least partially filled with a heat conduction fluid, or may be fluid-free. The bag is then permanently sealed. The particulates 200 may be filled with a heat absorbing fluid or solid. The particulates 200 may be solid, such as metallic to provide weighting. The particulates 200 may be a mixture of these, and of mixed sizes, to provide a predetermined distribution of weighting and predetermined distribution of heat absorption capacity. This exemplary embodiment may be used in conjunction with the multiple compartment exemplary embodiments described herein.

FIG. 8 illustrates a portion of an alternative embodiment of an exemplary “blister-pack-like” structure 800 that includes a series of structures, exemplified by an array of geometric-shaped cavities, exemplified by truncated pyramids 810 arrayed on a flexible plate 802, that is inserted into the bag 102 as a container to form the cold compress 800. The truncated pyramids (or other geometric shape) may be formed along with the plate; i.e. may be integral with the plate, as in blister-packs. The exemplary truncated pyramids 810 may be filled with a heat absorbing material that is then sealed into the cavity by sealing the cavity opening with a cover such as a film of suitable polymer. Some of the truncated pyramids 810 may be filled with weighting elements, such as beads of metal. This may assist in conforming the cold compress to human body curvatures and provide a satisfying weighted feel. The exemplary embodiment allows a cold compress to maintain a predetermined distribution of heat absorption capacity along with a weighted feel. This exemplary embodiment may be used in conjunction with the multiple compartment exemplary embodiments described herein.

While the invention has been described with respect to exemplary embodiments, other variants may be apparent to a person of ordinary skill in the art who has read this disclosure of technology. These variants are deemed within the scope of the appended patent claims and their equivalents.

We claim:

1. A cold compress used for therapeutic cooling comprising:
a permanently sealed flexible elastic bag comprised of a water and water vapor impermeable film, an interior volume of the sealed flexible elastic bag containing a plurality of free-flowing spheres, each of the plurality of free-flowing spheres having a surrounding outer layer and containing a material having a freezing point of about 0° C.;
wherein, when the cold compress is in use, the material absorbs latent heat to convert from a frozen state to a liquid state and the interior volume of the sealed bag is free of bridging between the plurality of spheres;
wherein, when the cold compress is in use, the cold sealed flexible elastic bag remains flexible and elastic, and the plurality of spheres remain free-flowing such that the cooled flexible sealed elastic bag conforms to a simply curved or compound curved surface of the human body and thereby presents a heat transfer contact surface area with the simply curved or compound curved surface of the body to absorb heat through the heat transfer contact surface area from the simply curved or compound curved surface of the body, the absorbed heat providing latent heat of liquefaction to frozen material inside the plurality of spheres such that a temperature of the cold sealed flexible elastic bag is substantially maintained until a substantial proportion of the frozen material has liquefied.

2. The cold compress of claim 1, wherein the material inside each of the plurality of spheres comprises wax.

3. The cold compress of claim 1, wherein the material inside each of the plurality of spheres comprises water.

4. The cold compress of claim 1, wherein said plurality of spheres are each in the size range from about 3 to about 25 mm.

5. The cold compress of claim 1, wherein the interior of the bag contains a substance that is liquid, non-reactive with the plurality of spheres, non-reactive with the flexible elastic sealed bag, and is not a solid in the range from about 0° C. to about −25° C.

6. The cold compress of claim 5, wherein the substance inside the sealed elastic flexible bag comprises a gel.

7. The cold compress of claim 5, wherein the substance inside the sealed elastic flexible bag comprises a solution of salt in water that remains liquid when the material inside the each of the plurality of spheres has frozen.

8. The cold compress of claim 1, wherein the cold compress contains a plurality of weighting elements.

9. The cold compress of claim 1, wherein at least some of the plurality of spheres are tethered to an inner surface of the permanently sealed flexible elastic bag.

10. The cold compress of claim 1, wherein the sealed elastic flexible bag has multiple internal compartments, each compartment containing some of the plurality of spheres.

11. The cold compress of claim 10, wherein dividers between the multiple internal compartments have through holes for fluid communication between compartments.

12. The cold compress of claim 10, wherein dividers between the multiple internal compartments allow no fluid communication between compartments.

13. The cold compress of claim 10, wherein at least some of the multiple internal compartments contains a substance that is liquid, non-reactive with the plurality of spheres, and non-reactive with the flexible elastic sealed bag.

14. The cold compress of claim 10, wherein a region around each of the dividers between the multiple internal compartments is flexible to facilitate folding the cold compress around simple curved or compound curved human body parts.

15. The cold compress of claim 14, further comprising weighting elements in at least some of the multiple compartments of the bag.

16. The cold compress of claim 1, wherein the plurality of spheres are distributed in a pattern by fixed attachment to a flexible structure that is sized and configured to fit inside the flexible elastic bag.

17. The cold compress of claim 16, wherein the structure has a net-like configuration, and the spheres are attached at predetermined points on the net-like structure.

18. The cold compress of claim 10, wherein at least some of the plurality of spheres are tethered to an inner surface of the multiple internal compartments.

19. The cold compress of claim 10, further comprising weighting elements tethered to an inner surface of the multiple internal compartments of the bag.

20. The cold compress of claim 10, wherein the plurality of spheres are distributed in a pattern by fixed attachment to a structure that is sized and configured to fit inside the internal compartments of the flexible elastic bag.

21. The cold compress of claim 20, wherein the structure has a net-like configuration, and the spheres are attached at predetermined points on the net-like structure.

22. A cold compress used for therapeutic cooling comprising:
a permanently sealed flexible elastic bag comprised of a water and water vapor impermeable film, an interior volume of the sealed flexible elastic bag containing a structure comprised of a flexible plate having arrayed therein a plurality of geometric shaped cavities, each having a volumetric capacity, the volumetric capacity containing sealed therein a material having a freezing point;
wherein, when the cold compress is in use, the material absorbs latent heat to convert from a frozen state to a liquid state; and
wherein, when the cold compress is in use, the cold sealed flexible elastic bag remains flexible and elastic, and the plate remains flexible so that the cold compress conforms to a simply curved or compound curved surface of the human body and thereby presents a heat transfer contact surface area with the simply curved or compound curved surface of the body to absorb heat through the heat transfer contact surface area from the simply curved or compound curved surface of the body.

23. The cold compress of claim 22, wherein some of the plurality of geometric shapes has a volumetric capacity filled with a weighting down medium sealed therein.

24. The cold compress of claim 22, wherein the permanently sealed flexible elastic bag is divided into multiple internal compartments and at least some of the compartments contains therein the structure comprised of a flexible plate having arrayed therein a plurality of geometric shapes.
